(19) United States
(12) Patent Application Publication
(10) Pub. No.: US 2015/0257697 A1
(43) Pub. Date: Sep. 17, 2015

(54) Method and System for Mobile, Social, Behavioral Treatment of Sleep

(71) Applicant: Saviz Sepah, San Diego, CA (US)

(72) Inventor: Saviz Sepah, San Diego, CA (US)

(21) Appl. No.: 14/215,312

(22) Filed: Mar. 17, 2014

(51) Int. Cl. A61B 5/00

(52) U.S. Cl. CPC A61B 5/486; A61B 5/4815; A61B 5/0022; A61B 5/746; A61B 5/7275; A61B 5/7455; A61B 5/7203; A61B 5/4833; A61B 5/7465; A61B 5/6824

(57) ABSTRACT

A method and system for mobile behavioral treatment of sleep issues such as insomnia comprising of: placing, participants into an online software platform that includes an online coach and group/community to reinforce compliance and provide social support; providing, a curriculum, compromising of modules of evidence-based behavioral treatments (e.g. cognitive-behavioral therapy (CBT), intensive sleep retraining (ISR)); providing, a wireless wearable body metric measurement device configured to communicate remotely with a mobile computing device and network; receiving a set of body metric measurement data via a mobile computing device; transmitting and storing the body metric measurement data on a server; determining trends and changes in the body metric measurement of the participant; providing, visual feedback regarding sleep quantity and quality to the participant via an online software platform that is accessible through mobile devices; calculating individualized recommendations based on body metric measurements and CBT protocols; providing, behavioral alerts to the participant via a wireless body metric device to alter sleep behaviors.

### CROSS-REFERENCE TO RELATED APPLICATIONS

This application claims the benefit of U.S. Provisional Application No. 61/792,012, titled “Mobile App for Behavioral Treatment of Sleep” filed on Mar. 15, 2013, which is incorporated in its entirety by this reference,

### FIELD

This invention relates generally to the medical field, and more specifically to an improved method for providing behavioral treatment for sleep issues such as insomnia in the medical field.

### BACKGROUND

Insomnia is characterized by a difficulty initiating or maintaining sleep, or experiencing non-restorative/refreshing sleep. Approximately 30% of adults report insomnia symptoms, 90% are undiagnosed, and 40% are diagnosed and untreated. The current gold standard, evidence-based. treatment for insomnia is cognitive-behavioral therapy (CBT).

CBT for insomnia is a multi-component treatment that includes documented behavioral techniques such as sleep hygiene, stimulus control, sleep restriction, and restructuring cognitive distortions. Newer variations of CBT for insomnia include intensive sleep retraining (ISR), which uses a combination of sleep restriction and repeatedly awakening a participant in order to accumulate sleep deprivation and behaviorally condition rapid sleep onset. CBT and its variations are typically provided by a professional in the context of therapy, or can be self-directed through workbooks or guided online programs.

However, existing solutions require provider oversight, non-passive measurement of steep parameters, or self-initiated behavior change, which severely limits treatment access and compliance. Thus, this invention addresses these issues using a mobile-based, online social software platform, using passive sleep measurement, and an automated behavioral feedback system to improve the accessibility and compliance with behavioral treatment for sleep disorders such as insomnia.

To the knowledge of the present inventor, the combination of a wireless wearable body metric measurement device (e.g. bluetooth-based watch/bracelet/pad/sensor), which transmits data to a mobile computing device (e.g. iOS/Android-based smart phone), which contains an online software platform to provide social support and CBT curriculum, and computes and transmits back to the wearable device individualized recommendations and behavioral alerts, has not been used or suggested. Examples of patents/devices used to provide automated behavioral treatment for insomnia (but do not specifically integrate wireless wearable body metric devices with existing mobile devices or use online software platforms to reinforce compliance and provide social support), include:

1) Kaplan et al. U.S. Pat. No. 8,512,221 B2 (Automated treatment system for sleep);

2) Naujokat et al. US 20120238800 A1 (Method and system for providing behavioural therapy for insomnia)

### BRIEF DESCRIPTION OF THE FIGURES

FIG. 1 is an example of a wireless wearable body metric measurement device (102) that passively tracks sleep parameters.

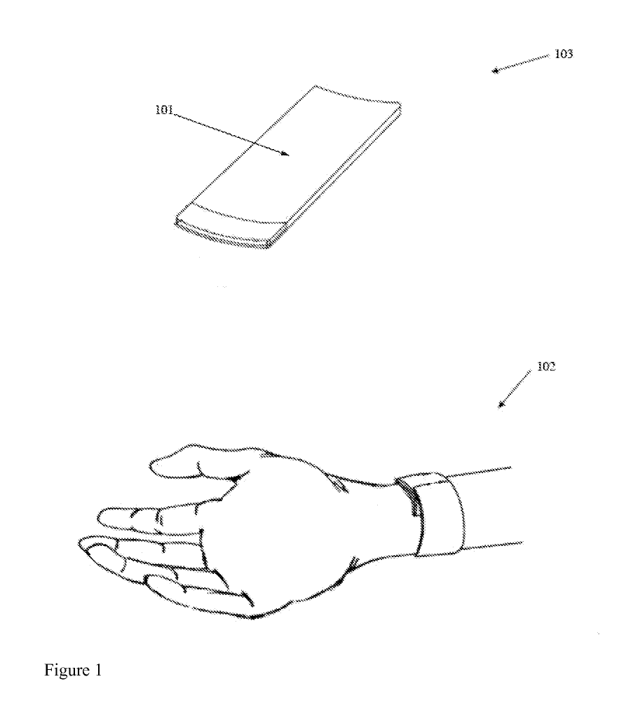

FIG. 2. Is an example of a mobile computing device (101), which acts as the hardware platform and a wireless receiver/transmitter.

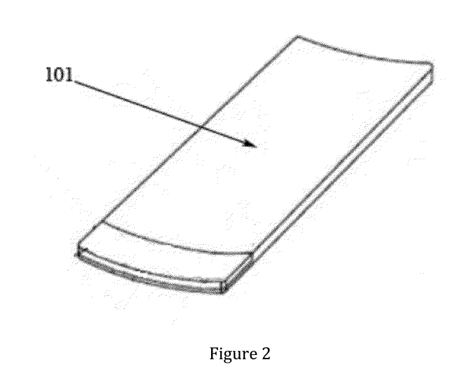

FIG. 3. Is an example of an online software platform (103), which provides CBT curriculum along with coaching/group social support, computes and displays sleep parameters, and provides individualized recommendations back to the wireless wearable device in the form of behavioral alerts.

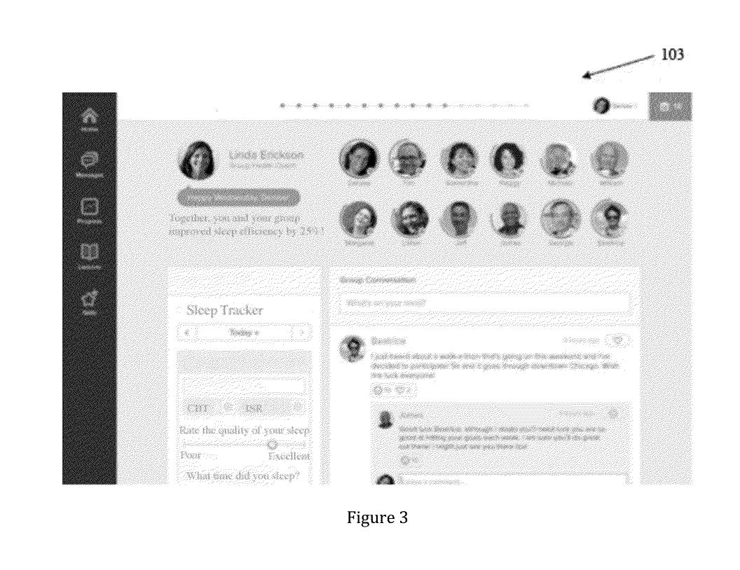

### SUMMARY OF THE INVENTION

The following description of embodiments of the invention is not intended to limit the invention to these embodiments, but rather to enable any person skilled in the art to make and use this invention.

A participant can be referred to the program if they have a self-reported or diagnosed sleep issue such as insomnia. The participant is placed into an online software platform that is accessible through web or mobile devices. The software comprises of a structured training program based on principles of cognitive-behavioral therapy (CBT) for insomnia. For example, the participant would complete a time-based curriculum (e.g. weekly lessons) that teaches the components of CBT, including sleep hygiene, stimulus control, sleep restriction, and restructuring cognitive distortions. An online coach (either an automated persona or a human health coach) would monitor progress and provide individualized feedback to the participant. An online group or community would provide social support and accountability to the participants to enhance treatment engagement. The software platform would also provide a system of behavioral reinforcement via “gamification” (e.g. rewarding achievement of behavioral goals (such as completing weekly lessons, participating in group discussion, tracking sleep metrics, and making changes to sleep habits) to enhance treatment compliance.

The participant would use a wireless wearable body metric measurement device to track sleep parameters. This device would most likely take the form of a watch/bracelet/pad/sensor e.g. worn on the wrist, chest, or head) that can comfortably be worn to sleep. The device would use body metric measurement (e.g. body movement/actigraphy, heart rate, heart rate/rhythm variability, electroencephalography (EEG), electromyography (EMG), peripheral arterial tone (PAT), systolic upstroke time, electrocardiography (ECG/EKG), electrooculography (EOG), oximetry, galvanic skin response (GSR), respiratory variability, eye movements, and or any combinations thereof). The device would transmit this data to a mobile computing device using wireless technology (e.g. Bluetooth, Near Field Communication (WC), or Cellular signals).

The participant would use an existing mobile computing device (e.g. an iOS or Android based. mobile smart phone or tablet) to download the online software platform to treat sleep. The mobile computing device would use its built-in wireless receiver to receive the data from the wireless wearable device. The online software platform would transmit this data to a server that would store, filter, and analyze this data. The software platform would then calculate standardized parameters of sleep quantity and quality (e.g. sleep onset latency, number and length of awakenings, total sleep time, wake time, overall sleep efficiency, and sleep restfulness/restorativeness). The online software platform would visualize that data to the participant to provide understandable feedback on their sleep quantity and quality. The software would use CBT guidelines to make individualized recommendations (e.g. prescribing the time to go to bed or get out of bed, when to get out of bed if unable to fall asleep, when to try to sleep again). The software would transmit these recommendations back to the wireless wearable device, where they would take the form of behavioral alerts (e.g. a vibration to go to bed or get out of bed) to reinforce behavioral compliance.

As a person skilled in the art will recognize from the previous detailed description and from the figures and claims, modifications and changes can be made to the preferred embodiments of the mention without departing from the scope of this mention defined in the following claims.

I claim:

1. A method and system for mobile behavioral treatment of sleep issues such as insomnia comprising of:
placing, participants into an online software platform that includes an online coach and group/community to reinforce compliance and provide social support;
providing, a curriculum, compromising of modules of evidence-based behavioral treatments (e.g. cognitive-behavioral therapy (CBT), intensive sleep retraining (ISR));
providing, a wireless wearable body metric measurement device configured to communicate remotely with a mobile computing device and network;
receiving a set of body metric measurement data via a mobile computing device;
transmitting and storing the body metric measurement data on a server;
determining trends and changes in the body metric measurement of the participant;
providing, data feedback regarding sleep quantity and quality to the participant via an online software platform that is accessible through mobile devices;
calculating individualized recommendations based on body metric measurements and evidence-based protocols such as CBT;
providing, behavioral alerts to the participant via a wireless body metric device to alter sleep behaviors.

2. The method of claim 2, wherein providing a CBT-based curriculum comprises a time series of evidence-based modules such as sleep hygiene, stimulus control, sleep restriction, restructuring cognitive distortions, and intensive sleep retraining.

3. The method of claim 1, wherein participants are placed into online groups or communities with shared health issues and goals to provide social support.

4. The method of claim 1, wherein participants are assigned an automated or human health coach to provide feedback and reinforce treatment compliance.

5. The method of claim 1, wherein providing, to each participant, a body metric measurement device, comprises providing a wireless wearable body metric measurement device configured to measure sleep parameters.

6. The method of claim 1, wherein participants download a mobile app, consisting of an online software platform to treat sleep issues, onto an existing mobile computing device (e.g. iOS/Android smart phone or tablet).

7. The method of claim 1, wherein the wearable wireless device transmits a set of time series body metric measurement data to the mobile computing device and the network.

8. The method of claim 7, wherein the set of time series of body metric measurement data is computed and visualized by the online software platform into parameters of sleep quantity and quality, and provides individualized feedback based on CBT.

9. The method of claim 8, wherein the online software platform uses sleep parameters to triggers behavioral alerts back to the wireless wearable device in the form of vibrations.

10. The method of claim 1, wherein storing the set of body metric measurement data comprises filtering the set of body metric measurement data to generate a filtered set of body metric measurement data.

11. The method of claim 10, wherein filtering the set of body metric measurement data comprises identifying an outlier or erroneous measurement that deviates from an adjacent measurement or line fitted to a time series by a threshold amount.

12. The method of claim 1, wherein determining a body metric measurement trend of the participant comprises determining a difference between a body metric measurement of the participant and an initial baseline body metric measurement of the participant (e.g. sleep parameters before and during/after treatment)

13. The method of claim 1, wherein determining a body metric measurement trend of the participant comprises determining a difference between a body metric measurement of the participant and an average of body metric measurements of the participant.

14. The method of claim 1, wherein determining a body metric measurement trend of the participant comprises determining a rate of progress.

15. The method of claim 1, wherein determining a body metric measurement trend of the portion of the matched group comprises determining a trend based on a subset of the set of body metric measurement data, wherein the subset excludes body metric measurements from the participant.

16. The method of claim 1, wherein providing feedback to the participant comprises enabling communication between a coach and the participant.

17. The method of claim 1, wherein providing feedback to the participant comprises enabling communication between the participant and a second participant.

18. The method of claim 1, further comprising providing behavioral reinforcement and rewards to the participant to enhance treatment engagement and compliance.

19. The method of claim 18, wherein providing behavioral reinforcement and rewards to the participant is based upon a performance metric of the participant.

20. The method of claim 19, wherein the performance metric is based on achievement of behavioral goals (e.g. completing weekly lessons, participating in group discussion, tracking sleep metrics, and making changes to sleep habits)
